(19) United States
(12) Patent Application Publication
(10) Pub. No.: US 2026/0363807 A1
(43) Pub. Date: Nov. 19, 2026

(54) DETERMINATION OF A CURVILINEAR DISTANCE WITHIN A SUBJECT

(71) Applicant: Intuitive Surgical Operations, Inc., Sunnyvale, CA (US)

(72) Inventors: Gaurav Lamba, Fremont, CA (US); Kenneth K. Lee, Oakton, VA (US)

(21) Appl. No.: 19/487,674

(22) PCT Filed: Jul. 3, 2024
(86) PCT No.: PCT/US2024/036784

§ 371 Date: Nov. 25, 2025

Related U.S. Application Data
(60) Provisional application No. 63/525,297, filed on Jul. 6, 2023.

(51) Int. Cl. A61B 34/00; A61B 34/20

(52) U.S. Cl. CPC A61B 34/70; A61B 34/20; A61B 2034/2065

(57) ABSTRACT

An illustrative dynamic measurement system may be configured to determine, based on positions of instruments within a scene, positions of points on anatomy in the scene, determine distance values representative of physical distances between sequential points, and determine a cumulative measurement value representative of a distance along the anatomy between the points. The cumulative measurement value may dynamically update as points are determined within the scene.

### RELATED APPLICATIONS

This application claims priority to U.S. Provisional Patent Application No. 63/525,297, filed Jul. 6, 2023, the contents of which is hereby incorporated by reference in its entirety.

### BACKGROUND INFORMATION

During a medical procedure, such as a procedure that utilizes a computer-assisted medical system, an imaging device may be used to provide (e.g., to a surgeon) images of internal anatomy within a subject. In some scenarios, it may be desirable to measure various distances associated with the internal anatomy for the procedure. For example, it may be desirable to measure a size of a bowel during certain bariatric and/or colorectal procedures.

Unfortunately, direct physical access to the internal anatomy may not be available (e.g., during a minimally invasive medical procedure). Moreover, the anatomy may be curved and/or folded in some portions, which may render measurements associated with anatomy difficult and/or time-consuming.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its purpose is to present one or more aspects of the systems and methods described herein as a prelude to the detailed description that is presented below.

An illustrative computer-assisted medical system includes a first repositionable manipulator arm coupled to a first instrument and configured to move the first instrument relative to an anatomy within a scene being imaged by an imaging device and a second repositionable manipulator arm coupled to a second instrument and configured to move the second instrument relative to the anatomy within the scene. The system further includes a computing device configured to perform a set of processes comprising: determining, based on a position of the first instrument within the scene, a position of a first point on the anatomy; determining, based on a position of the second instrument within the scene, a position of a second point on the anatomy; determining, based on the position of the first point and the position of the second point, a first distance value representative of a physical distance between the first point and the second point; identifying, based on an updated position of the second instrument, a position of a third point on the anatomy; determining, based on the position of the third point, a second distance value representative of a physical distance between the third point and the second point; and determining, based on the first and second distance values, a cumulative measurement value representative of a distance along the anatomy between the first, second, and third points.

An illustrative method includes determining, by a cumulative measurement system and based on a position of a first instrument within a scene, a position of a first point on an anatomy; determining, by the cumulative measurement system and based on a position of a second instrument within the scene, a position of a second point on the anatomy; determining, by the cumulative measurement system and based on the position of the first point and the position of the second point, a first distance value representative of a physical distance between the first point and the second point; identifying, by the cumulative measurement system and based on an updated position of the second instrument, a position of a third point on the anatomy; determining, by the cumulative measurement system and based on the position of the third point, a second distance value representative of a physical distance between the third point and the second point; and determining, by the cumulative measurement system and based on the first and second distance values, a cumulative measurement value representative of a distance along the anatomy between the first, second, and third points.

An illustrative non-transitory computer-readable medium may store instructions that, when executed, direct a processor of a computing device to perform a process comprising: determining, based on a position of a first instrument within a scene, a position of a first point on an anatomy; determining, based on a position of a second instrument within the scene, a position of a second point on the anatomy; determining, based on the position of the first point and the position of the second point, a first distance value representative of a physical distance between the first point and the second point; identifying, based on an updated position of the second instrument, a position of a third point on the anatomy; determining, based on the position of the third point, a second distance value representative of a physical distance between the third point and the second point; and determining, based on the first and second distance values, a cumulative measurement value representative of a distance along the anatomy between the first, second, and third points.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative computer-assisted medical system.

FIG. 2 shows an illustrative implementation of a cumulative measurement system.

FIG. 3 shows an illustrative method of operating a cumulative measurement system.

FIGS. 4A and 4B show illustrative implementations of determining a first distance value using a cumulative measurement system.

FIGS. 5A and 5B show illustrative implementations of determining a second measurement value using a cumulative measurement system.

FIG. 6 shows an illustrative implementation of a display that may be generated by a cumulative measurement system.

FIG. 7 shows an illustrative method of operating a cumulative measurement system.

FIGS. 8A and 8B show illustrative implementations of displays that may be generated by a cumulative measurement system.

FIGS. 9A and 9B show illustrative implementations of displays that may be generated using a cumulative measurement system.

FIGS. 10A and 10B show illustrative implementations of displays that may be generated by a cumulative measurement system.

FIG. 11 shows an illustrative implementation of a display that may be generated using a cumulative measurement system.

FIG. 12 shows an illustrative computing system according to principles described herein.

### DETAILED DESCRIPTION

An illustrative cumulative measurement system may be configured to provide an intraoperative cumulative measurement of a curvilinear distance associated with an anatomy by determining a sum of distances between a plurality of points identified on the anatomy depicted within a scene (e.g., a workspace within a subject of a medical procedure).

For example, the cumulative measurement system may be configured to determine positions of a first point and a second point on the anatomy, such as based on positions of a first instrument and a second instrument within a scene. For example, the first and second points may be determined based on the first and second instruments holding a portion of the anatomy between the first and second instruments (e.g., to straighten a portion of the anatomy that may be curved between the first and second instruments). Based on the positions of the first point and the second point, the cumulative measurement system may be configured to determine a first distance value representative of a physical distance between the first point and the second point.

The cumulative measurement system may further be configured to identify a position of a third point on the anatomy, such as based on an updated position of the second instrument. For example, the second instrument may be moved to the updated position relative to the anatomy (e.g., to hold a different portion of the anatomy between the first and second instruments such that the third point is identified outside of the region between the first and second points). Based on the position of the third point, the cumulative measurement system may be configured to determine a second distance value representative of a physical distance between the third point and the second point. The cumulative measurement system may further be configured to determine, based on the first and second distance values, a cumulative measurement value representative of a distance along the anatomy between the first, second, and third points.

In some implementations, positions of additional points may be determined on the anatomy (e.g., based on movement of the first instrument and/or second instrument) such that the cumulative measurement system may dynamically update the cumulative measurement value in real-time as sequential additional points are added. The cumulative measurement system may further be configured to track the positions of the determined points within the scene (e.g., as the anatomy moves within the scene due to interactions with the instruments or patient movements), which may use an endpoint of a previous distance value as a start point for a subsequent distance value (e.g., as the cumulative measurement value is updated).

The principles described herein result in improved cumulative measurements compared to conventional techniques that are not determined using a cumulative measurement system, as well as provide other benefits as described herein. For example, the cumulative measurement system may determine various distances (e.g., lengths, widths, circumferences, etc.) associated with portions of the anatomy that may be curved and/or folded more accurately and/or efficiently. To illustrate, the cumulative measurement system may determine positions of points on the anatomy within a scene (e.g., based on positions of first and second instruments relative to the anatomy) and determine the cumulative measurement value based on distances of several linear segments between the positions of points on the anatomy. The cumulative measurement system may further account for movement of the positions of the points and/or anatomy during the determination of the cumulative measurement value, which may decrease and/or eliminate factors (e.g., alignment of the segments, accurate measuring of the segments, accurate totaling of the segments, etc.) associated with determining distances of the anatomy. Moreover, the cumulative measurement system may dynamically update the cumulative measurement value (e.g., as additional points are determined), which may indicate when a desired measurement is reached.

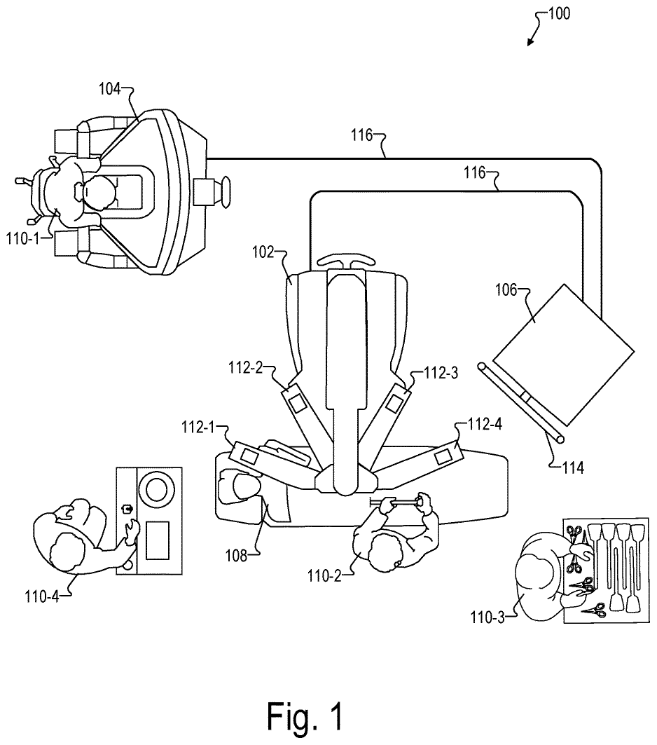

FIG. 1 shows an illustrative computer-assisted medical system 100 that may be used to perform various types of medical procedures including surgical and/or non-surgical procedures.

As shown, computer-assisted medical system 100 may include a manipulator assembly 102 (a manipulator cart is shown in FIG. 1), a user control apparatus 104, and an auxiliary apparatus 106, all of which are communicatively coupled to each other. Computer-assisted medical system 100 may be utilized by a medical team to perform a computer-assisted medical procedure or other similar operation on a body of a patient 108 or on any other body as may serve a particular implementation. As shown, the medical team may include a first user 110-1 (such as a surgeon for a surgical procedure), a second user 110-2 (such as a patient-side assistant), a third user 110-3 (such as another assistant, a nurse, a trainee, etc.), and a fourth user 110-4 (such as an anesthesiologist for a surgical procedure), all of whom may be collectively referred to as users 110, and each of whom may control, interact with, or otherwise be a user of computer-assisted medical system 100. More, fewer, or alternative users may be present during a medical procedure as may serve a particular implementation. For example, team composition for different medical procedures, or for non-medical procedures, may differ and include users with different roles.

While FIG. 1 illustrates an ongoing minimally invasive medical procedure such as a minimally invasive surgical procedure, it will be understood that computer-assisted medical system 100 may similarly be used to perform open medical procedures or other types of operations. For example, operations such as exploratory imaging operations, mock medical procedures used for training purposes, and/or other operations may also be performed.

As shown in FIG. 1, manipulator assembly 102 may include one or more manipulator arms 112 (e.g., manipulator arms 112-1 through 812-4) to which one or more instruments may be coupled. The instruments may be used for a computer-assisted medical procedure on patient 108 (e.g., in a surgical example, by being at least partially inserted into patient 108 and manipulated within patient 108). While manipulator assembly 102 is depicted and described herein as including four manipulator arms 112, it will be recognized that manipulator assembly 102 may include a single manipulator arm 112 or any other number of manipulator arms as may serve a particular implementation. While the example of FIG. 1 illustrates manipulator arms 112 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 112 shown in FIG. 1.

During the medical operation, user control apparatus 104 may be configured to facilitate teleoperational control by user 110-1 of manipulator arms 112 and instruments attached to manipulator arms 112. To this end, user control apparatus 104 may provide user 110-1 with imagery of an operational workspace associated with patient 108 as captured by an imaging device. To facilitate control of instruments, user control apparatus 104 may include a set of master controls. These master controls may be manipulated by user 110-1 to control movement of the manipulator arms 112 or any instruments coupled to manipulator arms 112.

Auxiliary apparatus 106 may include one or more computing devices configured to perform auxiliary functions in support of the medical procedure, such as providing insufflation, electrocautery energy, illumination or other energy for imaging devices, image processing, or coordinating components of computer-assisted medical system 100. In some examples, auxiliary apparatus 106 may be configured with a display monitor 114 configured to display one or more user interfaces, or graphical or textual information in support of the medical procedure. In some instances, display monitor 114 may be implemented by a touchscreen display and provide user input functionality. Augmented content provided by a region-based augmentation system may be similar, or differ from, content associated with display monitor 114 or one or more display devices in the operation workspace (not shown).

Manipulator assembly 102, user control apparatus 104, and auxiliary apparatus 106 may be communicatively coupled to another in any suitable manner. For example, as shown in FIG. 1, manipulator assembly 102, user control apparatus 104, and auxiliary apparatus 106 may be communicatively coupled by way of control lines 116, which may represent any wired or wireless communication link as may serve a particular implementation. To this end, manipulator assembly 102, user control apparatus 104, and auxiliary apparatus 106 may each include one or more wired or wireless communication interfaces, such as one or more local area network interfaces, Wi-Fi network interfaces, cellular interfaces, and so forth.

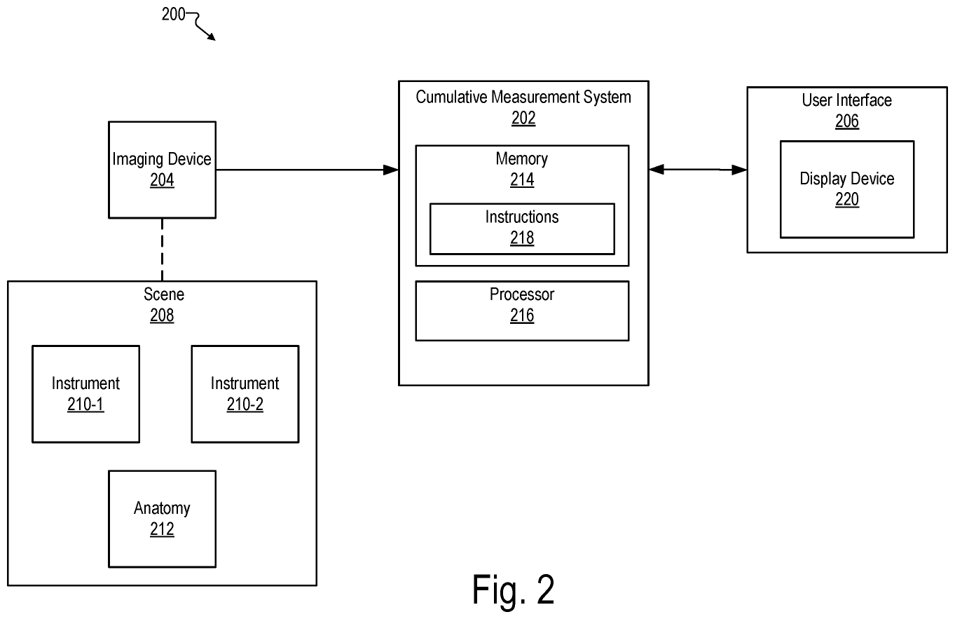

FIG. 2 shows an illustrative implementation 200 configured to determine a cumulative measurement value representative of a distance along anatomy within a scene. As shown, implementation 200 includes a cumulative measurement system 202 communicatively coupled (e.g., wired and/or wirelessly) with an imaging device 204 and a user interface 206. Implementation 200 may include additional or alternative components as may serve a particular implementation. In some examples, implementation 200 or certain components of implementation 200 may be implemented by a computer-assisted medical system (e.g., computer-assisted medical system 100).

Imaging device 204 may be implemented by an endoscope or other suitable device configured to capture images (e.g., images, videos, a sequence of image frames, etc.) of a scene 208. In some implementations, imaging device 204 may include, but is not limited to, one or more of: video imaging devices, infrared imaging devices, visible light imaging devices, non-visible light imaging devices, intensity imaging devices (e.g., color, grayscale, black and white imaging devices), depth imaging devices (e.g., stereoscopic imaging devices, time-of-flight imaging devices, infrared imaging devices, red-green-blue (RGB) imaging devices, red-green-blue and depth (RGB-D) imaging devices, light detection and ranging (LIDAR) imaging devices, etc.).

In some implementations, the images include image data (e.g., color, grayscale, saturation, intensity, brightness, depth, etc.) captured by imaging device 204. The image data may, in some instances, be associated with data points expressed in a common coordinate frame such as three-dimensional (3D) voxels or two-dimensional (2D) pixels of images captured by imaging device 204. In some implementations, imaging device 204 may be moved relative to scene 208 to capture images of scene 208 at different viewpoints.

Scene 208 includes an environment (e.g., a workspace within a subject of a medical procedure) and one or more objects within the environment. For example, scene 208 includes a plurality of instruments 210 (e.g., instruments 210-1 through 210-2) and anatomy 212. For example, instruments 210 may include physical tools (e.g., scalpels, scissors, forceps, clamps, etc.) that may be configured to manipulate the anatomy 212 within scene 208. In some implementations, instruments 210 may be coupled with manipulator arms (e.g., manipulator arms 112) of a computer-assisted medical system (e.g., computer-assisted medical system 100) such that the manipulator arms may be configured to move instruments 210 relative to anatomy 212.

To illustrate, a first repositionable manipulator arm may be coupled to a first instrument 210-1 and configured to move the first instrument 210-1 relative to anatomy 212 within scene 208 being imaged by imaging device 204. Additionally, a second repositionable manipulator arm may be coupled to a second instrument 210-2 and configured to move the second instrument 210-2 relative to anatomy 212 within scene 208. In some implementations, a third repositionable manipulator arm may be coupled to imaging device 204 and configured to move imaging device 204 to change a pose of imaging device 204 (e.g., relative to scene 208) such that imaging device 204 may capture images of scene 208 at different viewpoints.

Anatomy 212 includes one or more objects associated with a subject (e.g., a body of a live animal, a human or animal cadaver, a portion of human or animal anatomy, tissue removed from human or animal anatomies, non-tissue work pieces, training models, etc.). In some implementations, anatomy 212 may include tissue of a subject (e.g., an organ, soft tissue, connective tissue, etc.). In some implementations, at least a portion of anatomy 212 (e.g., a bowel, a stomach, etc.) may be curved within scene 208. Still other non-anatomical objects may be included within scene 208, such as objects (e.g., staples, mesh, sponges, etc.) used for a medical procedure.

Cumulative measurement system 202 is configured to receive or access images of scene 208 from imaging device 204. In some implementations, cumulative measurement system 202 may be configured to fuse imagery of scene 208 captured by imaging device 204 at different viewpoints of scene 208. In certain examples, the fusing may include merging aligned (or overlapping) voxels or pixels, such as by blending intensity and/or depth values for aligned voxels or pixels. The blending may include weighted blending in which the data points being blended are weighted based on one or more factors, such as which camera of a stereoscopic device has the best view of a data point (e.g., by more heavily weighting data captured by the camera with the best viewing angle). The fusing may additionally or alternatively include stitching non-overlapping voxels or pixels together, such as by stitching images together along non-overlapping boundaries of the images. Accordingly, the images of scene 208 may include an area that is larger than a single field of view of imaging device 204 based on the fusing of images at different viewpoints.

Based on the images of the scene, cumulative measurement system 202 is configured to determine a cumulative measurement value representative of a distance along anatomy 212 depicted within scene 208. For example, cumulative measurement system 202 may be configured to determine positions of points on anatomy 212 within scene 208, determine distance values representative of physical distances between sequential points, and determine, based on the distance values, a cumulative measurement value representative of a distance along anatomy 212 between the points. In some implementations, the distance along anatomy 212 may be curved such that the cumulative measurement value represents a curvilinear distance along anatomy 212. The cumulative measurement value may be represented by any suitable value, such as a discrete value (e.g., a distance, a range, a percentage, etc.) representative of the distance along anatomy 212.

Cumulative measurement system 202 may be implemented by one or more computing devices and/or computer resources (e.g., processors, memory devices, storage devices, etc.) as may serve a particular implementation. As shown, cumulative measurement system 202 may include, without limitation, a memory 214 and a processor 216 selectively and communicatively coupled to one another. Memory 214 and processor 216 may each include or be implemented by computer hardware that is configured to store and/or process computer software. Various other components of computer hardware and/or software not explicitly shown in FIG. 2 may also be included within cumulative measurement system 202. In some examples, memory 214 and/or processor 216 may be distributed between multiple devices and/or multiple locations as may serve a particular implementation.

Memory 214 may store and/or otherwise maintain executable data used by processor 216 to perform any of the functionality described herein. For example, memory 214 may store instructions 218 that may be executed by processor 216.

Memory 214 may be implemented by one or more memory or storage devices, including any memory or storage devices described herein, that are configured to store data in a transitory or non-transitory manner. Instructions 218 may be executed by processor 216 to cause cumulative measurement system 202 to perform any of the functionality described herein. Instructions 218 may be implemented by any suitable application, software, code, and/or other executable data instance. Additionally, memory 214 may also maintain any other data accessed, managed, used, and/or transmitted by processor 216 in a particular implementation.

Processor 216 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 216 (e.g., when processor 216 is directed to perform operations represented by instructions 218 stored in memory 214), cumulative measurement system 202 may perform various operations as described herein.

User interface 206 may include a display device 220 and may be configured to receive the cumulative measurement value from cumulative measurement system 202. Display device 220 may be implemented by a monitor or other suitable device configured to display information to a user. For example, display device 220 may be configured to display the cumulative measurement value received from cumulative measurement system 202. In some implementations, display device 220 may further be configured to display images of scene 208 captured by imaging device 204. Additionally or alternatively, user interface 206 may include any suitable device (e.g., a button, joystick, touchscreen, keyboard, handle, etc.) configured to receive a user input such as to identify and/or adjust positions of points within scene 208 for determining the cumulative measurement value.

In some implementations, cumulative measurement system 202 may be configured to determine multiple cumulative measurements within scene 208. As an illustrative example, cumulative measurement system 202 may be configured to determine a distance associated with a first portion of anatomy 212 within scene 208 and a distance associated with a second portion of anatomy 212 within scene 208.

Moreover, cumulative measurement system 202 may be configured to mark, track, and/or present the multiple cumulative measurements. For example, cumulative measurement system 202 may be configured to mark (e.g., highlight) anatomy 212, points identified on anatomy 212, and/or distances between the points on anatomy 212 (e.g., on a display of display device 220). Cumulative measurement system 202 may further be configured to track and update the multiple cumulative measurements as points are determined on anatomy 212. Cumulative measurement system 202 may further be configured to present (e.g., label) the multiple cumulative measurements to a user (e.g., on a display of display device 220).

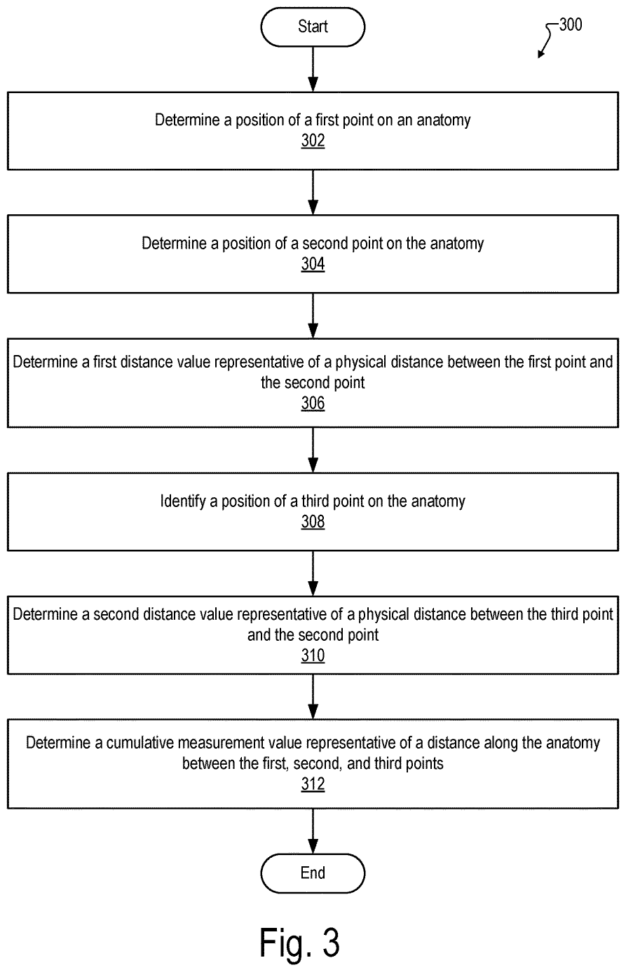

FIG. 3 shows an illustrative method 300 that may be performed by cumulative measurement system 202. While FIG. 3 illustrates operations according to one embodiment, other embodiments may omit, add to, reorder, and/or modify any of the operations shown in FIG. 3. Moreover, each of the operations depicted in FIG. 3 may be performed in any of the ways described herein.

As shown, cumulative measurement system 202 may, at operation 302, determine a position of a first point on an anatomy (e.g., anatomy 212) and, at operation 304, determine a position of a second point on the anatomy. Cumulative measurement system 202 may determine the position of the second point prior to, subsequent to, or concurrently with determining the position of the first point. Moreover, the position of the second point may be spaced a distance away from the position of the first point in the scene.

In some implementations, the first point is determined based on a position of a first instrument (e.g., first instrument 210-1) within a scene (e.g., scene 208) and the second point is determined based on a position of a second instrument (e.g., second instrument 210-2) within the scene. For example, the positions of the first and second points may be determined at and/or near positions of the first and second instruments relative to the anatomy within the scene. To illustrate, a portion of the anatomy may be held between the first and second instruments such that the positions of the first and second points may be determined on the anatomy at and/or near locations where the first and second instruments are holding the portion of the anatomy.

In some implementations, the positions of the first and second points may be determined based on positions of one or more features of the first and second instruments. For example, the first and second instruments may each include an instrument head (e.g., at a distal portion of the first and second instruments away from the manipulator arms) such that the positions of the points may be determined to correspond to the positions of the instrument heads of the first and second instruments. To illustrate, the first point may be aligned with a position of a feature (e.g., a central portion, a base, a tip, etc.) of the instrument head of the first instrument and the second point may be aligned with a position of a feature (e.g., a central portion, a base, a tip, etc.) of the instrument head of the second instrument. In some implementations, the points may be offset from the instrument heads at a select distance (e.g., between about 0.5 centimeters and about 2 centimeters, such as about 1 centimeter) within the scene. The select distance may, in some instances, be adjusted based on detecting a user input designating the select distance.

In some implementations, the positions of the first and second instruments may be determined based on kinematics data associated with the first and second instruments that may be representative of movement of the first and second instruments over time. In some implementations, the kinematics data may be generated by or associated with a computer-assisted medical system (e.g., computer-assisted medical system 100) communicatively coupled with the first and second instruments.

Additionally or alternatively, the positions of the first and second instruments may be determined based on a simultaneous localization and mapping (SLAM) heuristic. For example, a SLAM heuristic may be used by cumulative measurement system 202 to construct and/or update a map of the scene while simultaneously keeping track of the location of the first and second instruments (e.g., relative to the anatomy) within the scene. To illustrate, the SLAM heuristic may be configured to generate a point cloud having a plurality of nodes representative of surface points on one or more objects (e.g., the first and second instruments, the anatomy, etc.) within the scene as an imaging device (e.g., imaging device 204) views the scene in real-time. The 3D locations of the plurality of nodes may be derived from images (e.g., pixels and/or voxels of the images) captured by the imaging device. In instances where the plurality of nodes is based on 2D images, cumulative measurement system 202 may be configured to determine a depth associated with the plurality of nodes, such as by processing stereoscopic images captured by the imaging device.

The SLAM heuristic may further be configured to derive 3D locations of additional nodes as the imaging device is moved relative to the scene to capture additional regions of the scene, while also tracking the 3D locations of the previous nodes as one or more objects within the scene move. In some implementations, the SLAM heuristic may be configured to track a pose of the imaging device (e.g., using vision software) while the imaging device is moved relative to the scene.

Cumulative measurement system 202 may further be configured to track the positions of the determined points using the SLAM heuristic. As an illustrative example, the points may be associated with one or more nodes of the plurality of nodes generated using the SLAM heuristic such that the 3D locations of the points to be tracked with the 3D locations of the associated one or more nodes as the instruments and/or anatomy moves within the scene. Cumulative measurement system 202 may further be configured to track the positions of points while the points are outside of the field of view of the imaging device.

Additionally or alternatively, the positions of the first and second instruments may be determined based on a depth map of the scene. For example, the depth map of scene may be generated using a depth sensor and/or by processing stereoscopic images captured by the imaging device.

Additionally or alternatively, the positions of the first and second instruments may be determined by implementing and applying artificial intelligence algorithms, such as machine learning algorithms, to identify the first and second instruments in the scene. Any suitable form of artificial intelligence and/or machine learning may be used, including, for example, deep learning, neural networks, etc. For example, a machine learning algorithm may be generated through machine learning procedures and applied to identification operations. In some implementations, the machine learning algorithm may be directed to identifying instruments and/or a feature of the instruments within the scene. The machine learning algorithm may operate as an identification function that is applied to individual and/or fused imagery to classify instruments in the images.

Still other suitable methods may be used for determining the positions of the first and second instruments in addition to or instead of machine learning algorithms.

For example, cumulative measurement system 202 may be configured to identify the first and second instruments within the scene by implementing and applying object recognition algorithms. For example, an object recognition algorithm may be used to identify objects (e.g., instruments 210) of predetermined types within the image data received from the imaging device, such as by comparing the image data received from the imaging device to model object data of predetermined types of objects. Such model object data may be stored within a model database that may be communicatively coupled with cumulative measurement system 202.

Cumulative measurement system 202 may further, at operation 306, determine a first distance value representative of a physical distance between the first point and the second point. For example, the first distance value may be determined based on the positions of the first and second points such that cumulative measurement system 202 may determine the first distance value by determining a linear distance between the 3D location of the first point to the 3D location of the second point.

Cumulative measurement system 202 may further, at operation 308, identify a position of a third point on the anatomy. For example, the position of the third point may be identified based on an updated position of the second instrument. To illustrate, the second instrument may be moved relative to the anatomy to an updated position within the scene (e.g., to hold a different portion of the anatomy between the first and second instruments). The updated position of the second instrument within the scene may be determined by using one or more of kinematics data associated with the second instrument, a SLAM heuristic, a depth map of the scene, or a machine learning algorithm. In some implementations, the position of the third point may be identified on the anatomy at and/or near the updated position of the second instrument such that the position of the third point is spaced a distance away from the position of the second point within the scene.

Cumulative measurement system 202 may further, at operation 310, determine a second distance value representative of a physical distance between the third point and the second point. For example, the second distance value may be determined based on the position of the third point such that cumulative measurement system 202 may determine the second distance value by determining a linear distance between the 3D location of the third point to the 3D location of the second point.

Cumulative measurement system 202 may further, at operation 312, determine a cumulative measurement value representative of a distance along the anatomy between the first, second, and third points. For example, the cumulative measurement value may be determined as a sum of the first and second distance values. In some implementations, the points may be positioned along a curved portion of the anatomy such that the cumulative measurement value may provide an estimation of a curvilinear distance along the curved portion of the anatomy.

In some implementations, the cumulative measurement value may be dynamically updated such as while one or both of the instruments are being moved within the scene. To illustrate, prior to determining the position of the third point, the second instrument may move within the scene such that the distance between the second point and the second instrument changes as the second instrument moves. Cumulative measurement system 202 may dynamically update the second distance value, as well as the cumulative measurement value, as the distance between the second point and the second instrument changes. Once the position of the third point is determined (e.g., based on a designation by a user input), the second distance value and cumulative measurement value may be determined and stored (e.g., as static values based on the determined position of the third point). This process may be repeated as positions of additional points are determined. Because the distance values and cumulative measurement value are stored as positions of additional points are determined, the positions of previous points and/or segments between the positions of the previous points need not be within the view of the scene (e.g., while a distance value is being determined between positions of points shown within the scene) such that the positions of the previous points may move outside of the scene as positions of additional points are determined.

In some implementations, method 300 may further include performing, by cumulative measurement system 202, an operation based on the cumulative measurement value. For example, cumulative measurement system 202 may be configured to instruct one or more display devices (e.g., display device 220) to display the cumulative measurement value, the positions of points determined on the anatomy, and/or distance values associated with distances between points on the anatomy (e.g., overlaid on images depicting the scene).

Additionally or alternatively, the operation may include determining a size of the anatomy based on the cumulative measurement value. As an illustrative example, the cumulative measurement value may indicate a length of a bowel, which may be used to appropriately size a Roux limb associated with the length of the bowel. As another example, the cumulative measurement value may indicate a circumference of an interior surface of a stomach, which may be used to appropriately size a gastric band associated with the circumference. Still other operations based on the cumulative measurement value may be performed.

In some implementations, the cumulative measurement value may further be determined based on a user input. For example, cumulative measurement system 202 may be configured to detect a user input designating that a computer-assisted medical system (e.g., computer-assisted medical system 100) is in a measurement mode such that cumulative measurement system 202 may be configured to determine the positions of points and/or distance values based on detecting the user input to determine that the computer-assisted medical system is in the measurement mode.

To illustrate, the user input may include positioning one or both of the first and second instruments in a predetermined pose and/or performing a predetermined movement (e.g., a tap, a wave, and/or other gesture) with one or both of the first and second instruments. As an illustrative example, the first and second instruments may be positioned in the predetermined pose (e.g., for a predetermined amount of time) to hold a portion of the anatomy between the first and second instruments such that cumulative measurement system 202 may determine that the computer-assisted medical system is in the measurement mode and/or designate points on the anatomy relative to the positions of the first and second instruments in the predetermined pose. As another example, the first and second instruments may be moved to perform the predetermined movement such that cumulative measurement system 202 may determine that the computer-assisted medical system is in the measurement mode and/or designate points on the anatomy relative to where the predetermined movement was performed.

In some implementations, the user input may additionally or alternatively include a selectable option. For example, the selectable option (e.g., “determine measurement”) may be presented in a display (e.g., by display device 220) that may be selected by a user input (e.g., by user interface 206). Based on the user input, cumulative measurement system 202 may be configured to determine that the computer-assisted medical system is in the measurement mode and/or determine the positions of points based on the positions of the first and second instruments, such as at and/or near the time that the selectable option is selected.

Additionally or alternatively, the user input may include a voice command that may be received by cumulative measurement system 202 to determine that the computer-assisted medical system is in the measurement mode and/or determine the positions of points based on the positions of the first and second instruments, such as at and/or near the time that the voice command is received.

In some implementations, the user input may include a designation of positions of additional points on the anatomy within the scene (e.g., while the computer-assisted medical system in the measurement mode). For example, cumulative measurement system 202 may be configured to receive the user input designating the positions of the additional points by displaying images of the scene on a display device (e.g., display device 220). A designation of the positions of the additional points may be performed as a discrete event (e.g., a touch gesture, a button press, a mouse click, a button release, etc.) on any point of the images of the scene on the display device.

In some implementations, the cumulative measurement value may be adjusted based on a user input (e.g., while the computer-assisted medical system is in the measurement mode). For example, cumulative measurement system 202 may be configured to detect a user input designating the positions of one or more points on the anatomy within the scene to be adjusted. To illustrate, the user input may be performed as a discrete event (e.g., a touch gesture, a button press, a mouse click, a button release, etc.) on any point of the images of the scene on the display device such as to move and/or adjust the positions of the one or more points on the anatomy within the scene, which may cause cumulative measurement system 202 to update the distance values associated with the positions of the one or more points. The cumulative measurement value may further be updated based on the updated distance values.

In some implementations, cumulative measurement system 202 may be configured to determine when the measurement mode of the computer-assisted medical system has ended such as to stop determining the positions of points and/or distance values. For example, cumulative measurement system 202 may determine that the measurement mode has ended such as based on a user input (e.g., performing a predetermined movement and/or pose with the instruments, a voice command, a selectable option, etc.) designating the end of the measurement mode.

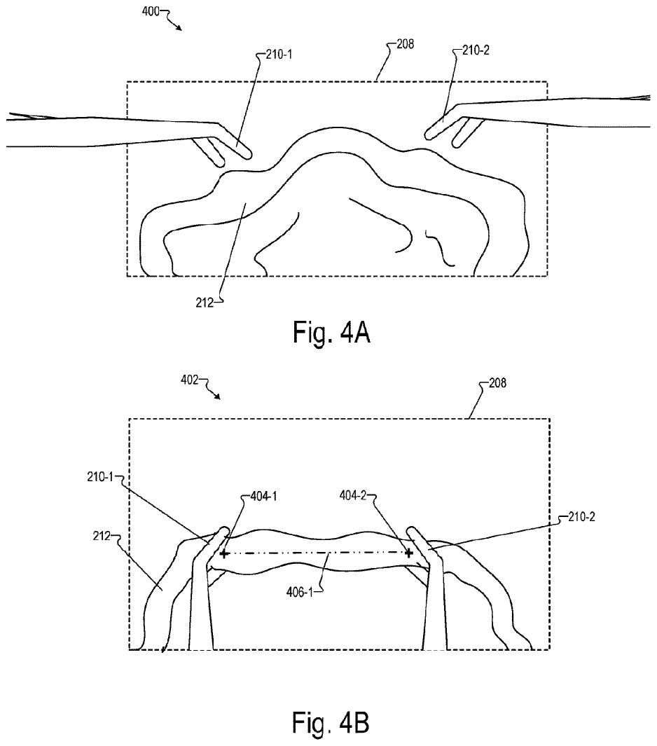

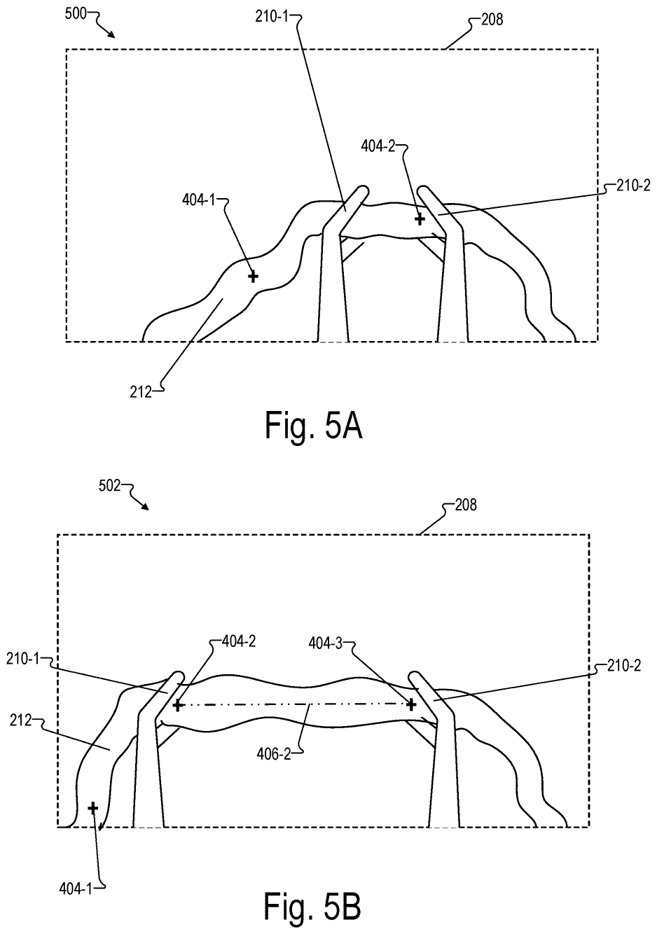

FIGS. 4A-5B show implementations of determining a cumulative measurement value by cumulative measurement system 202. For example, FIG. 4A shows an illustrative implementation 400 of scene 208 that may be captured by an imaging device (e.g., imaging device 204). As shown, scene 208 depicts a first instrument 210-1, a second instrument 210-2, and at least a portion of an anatomy 212. In the illustrated implementation, the portion of the depicted anatomy 212 within scene 208 is curved.

FIG. 4B shows an illustrative implementation 402 of the portion of anatomy 212 depicted in scene 208 being held between first instrument 210-1 and second instrument 210-2 such that the portion of anatomy 212 is straightened between first instrument 210-1 and second instrument 210-2. Positions of a first point 404-1 and a second point 404-2 are determined on the portion of anatomy 212 straightened between first instrument 210-1 and second instrument 210-2 (e.g., at and/or near the positions of instruments 210). For example, the position of first point 404-1 is offset from the position of a middle portion of an instrument head of first instrument 210-1 (e.g., towards second instrument 210-2) and the position of second point 404-2 is offset from the position of a middle portion of an instrument head of second instrument 210-2 (e.g., towards first instrument 210-1). A first distance value representative of a physical distance between first point 404-1 and second point 404-2 may be determined based on a linear segment 406-1 extending between the position of first point 404-1 and the position of second point 404-2.

FIG. 5A shows an illustrative implementation 500 of first and second instruments 210 being moved to updated positions. For example, first instrument 210-1 is moved toward second point 404-2 and second instrument 210-2 is moved along the portion of anatomy 212 away from second point 404-2 such as to hold a different portion of anatomy 212. Still other updated positions for first instrument 210-1 and/or second instrument 210-2 may be used. To illustrate, second instrument 210-2 may be moved away from second point 404-2 while first instrument 210-1 is not moved and/or first instrument 210-1 may be moved to an opposing side of second instrument 210-2 away from second point 404-2 while second instrument 210-2 is not moved. In some instances, the portion of anatomy 212 within scene 208 may move as one or both of instruments 210 are moved to the updated positions. In such instances, cumulative measurement system 202 may track the positions of points 404 on anatomy 212 within scene 208 as anatomy 212 moves.

FIG. 5B shows an illustrative implementation 502 of the different portion of anatomy 212 being held between first instrument 210-1 and second instrument 210-2 in the updated positions such that the different portion of anatomy 212 is straightened between first instrument 210-1 and second instrument 210-2. In the illustrated implementation, second point 404-2 remains positioned between first instrument 210-1 and second instrument 210-2 (e.g., closer to first instrument 210-1). A third point 404-3 is determined on the portion of anatomy 212 straightened between first instrument 210-1 and second instrument 210-2. For example, the position of third point 404-3 is offset from a middle portion of the instrument head of second instrument 210-2 (e.g., towards second instrument 210-2). A second distance value representative of a physical distance between second point 404-2 and third point 404-3 may be determined based on a linear segment 406-2 extending between second point 404-2 and third point 404-3. The cumulative measurement value may be determined based on the distance values associated with linear segments 406 such as by summing the distances of linear segments 406.

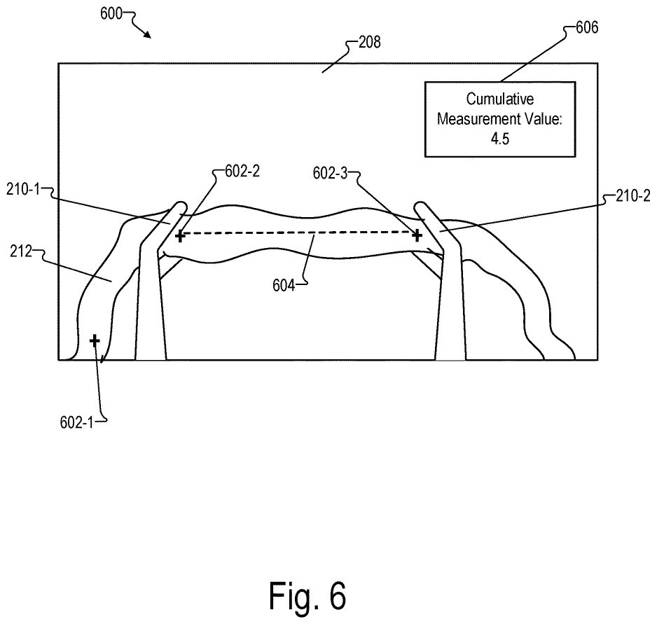

FIG. 6 shows an illustrative implementation 600 of a display including the cumulative measurement value that may be generated by cumulative measurement system 202 and displayed by a display device (e.g., display device 220). As shown, the display includes images of scene 208 depicting a portion of anatomy 212 being held between instruments 210. The display further includes a plurality of visual markers 602 (e.g., visual markers 602-1 through 602-3) representative of positions of points 404 on anatomy 212 within scene 208. For example, a first marker 602-1 represents the position of first point 404-1, a second marker 602-2 represents the position of second point 404-2, and a third marker 602-3 represents the position of third point 404-3. In some implementations, a visual marker 602 representing the position of a starting point (e.g., first point 404-1) of the cumulative measurement value and/or a visual marker 602 representing the position an end point (e.g., third point 404-3) of the cumulative measurement value may be unique relative to the other visual markers 602 representing positions of intermediate points (e.g., second point 404-2) of the cumulative measurement value.

In some implementations, the display may further include a virtual overlay 604 between sequential visual markers 602 within scene 208 representative of distances between the sequential visual markers 602. To illustrate, the virtual overlay 604 may be shown between visual markers 602 located on the portion of anatomy 212 being held between instruments 210. In some implementations, the display may further include a display of one or more distance values that may be representative of distances between the sequential visual markers 602 associated with virtual overlay 604.

Additionally or alternatively, the display may include a display of cumulative measurement value 606 (e.g., “4.5”). In some implementations, the display of cumulative measurement value 606 is updated as cumulative measurement system 202 updates cumulative measurement value 606 (e.g., as additional points 404 are determined within scene 208). For example, cumulative measurement value 606 may include a first distance value representative of the distance between first point 404-1 and second point 404-2 when first point 404-1 and second point 404-2 are determined. Cumulative measurement value 606 may then be updated to include a second distance value when third point 404-3 is added to include the distance between second point 404-2 and third point 404-3. Accordingly, a user may determine a distance associated with anatomy 212 based on cumulative measurement value 606 and/or determine when cumulative measurement value 606 reaches a desired distance. In some implementations, the display may further include a virtual overlay representative of cumulative measurement value 606 (e.g., along markers 602 within scene 208).

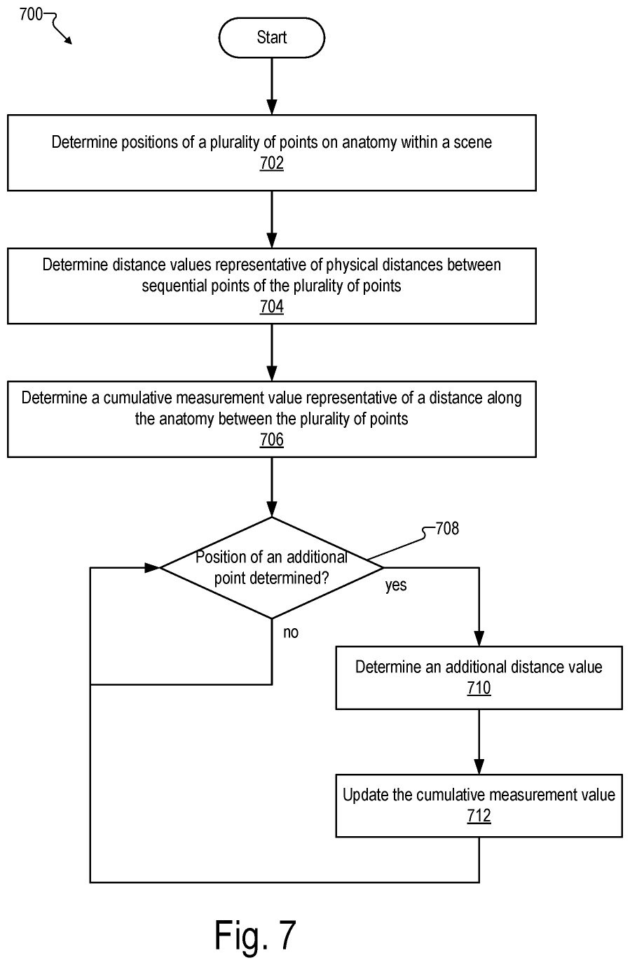

FIG. 7 shows another illustrative method 700 that may be performed by cumulative measurement system 202. While FIG. 7 illustrates operations according to one embodiment, other embodiments may omit, add to, reorder, and/or modify any of the operations shown in FIG. 7. Moreover, each of the operations depicted in FIG. 7 may be performed in any of the ways described herein.

As shown, cumulative measurement system 202 may, at operation 702, determine positions of a plurality of points (e.g., points 404) on anatomy (e.g., anatomy 212) within a scene (e.g., scene 208). For example, the positions of the points may be determined based on positions of one or more instruments (e.g., instruments 210) within the scene. For example, the positions of the points may be determined at and/or near the positions of the instruments while the instruments are holding at least a portion of the anatomy. In some implementations, the positions of the instruments within the scene may be determined based on at least one of kinematics data associated with the instruments, a SLAM heuristic, a depth map of the scene, or a machine learning algorithm.

Cumulative measurement system 202 may further, at operation 704, determine distance values representative of physical distances between sequential points of the plurality of points. For example, cumulative measurement system 202 may determine a first distance value representative of a physical distance between the positions of a first point and a second point. Cumulative measurement system 202 may further determine a second distance value representative of a physical distance between the positions of the second point and a third point. In some implementations, cumulative measurement system 202 may be configured to determine the distance values based on detecting when the instruments are holding a portion of the anatomy between the instruments. To illustrate, the first distance value may be determined while the instruments are holding the portion of the anatomy on which the first and second points are positioned and the second distance value may be determined while the instruments are holding the portion of the anatomy on which the second and third points are positioned.

Additionally or alternatively, the distance values may be determined based on the tracking the points within the scene. For example, in some instances, the positions of the instruments may move while the instruments are holding the portion of the anatomy between the instruments such that the anatomy and/or points positioned on the anatomy may correspondingly move with the instruments. This may cause the distances between the points positioned on the portion of the anatomy being held between the instruments to change such that the distance value associated with the points positioned on the anatomy may correspondingly change. The distance value may be dynamically updated based on the tracked positions of the points on the anatomy between the instruments. In some implementations, the distance value may be determined when the positions of the instruments remain still (e.g., for a predetermined amount of time) and/or based on a user input (e.g., designating the distance value to be determined).

Cumulative measurement system 202 may further, at operation 706, determine a cumulative measurement value representative of a distance along the anatomy between the plurality of points. For example, cumulative measurement system 202 may calculate the cumulative measurement value as a sum of the determined distance values.

In some implementations, positions of additional points may be determined on the anatomy within the scene (e.g., after the cumulative measurement value has been determined). For example, the first and/or second instruments may be moved to updated positions along the anatomy to designate positions of additional points on the anatomy. Additionally or alternatively, a user input may be detected that designates positions of additional points on the anatomy within the scene. Accordingly, cumulative measurement system 202 may, at operation 708, determine whether a position of an additional point has been determined on the anatomy.

If a position of an additional point has been determined (yes, at operation 708), cumulative measurement system 202 may, at operation 710, determine an additional distance value representative of a physical distance between the position of the additional point and the position of a previous point. For example, an endpoint of a previous distance value may be used as a start point for a subsequent additional distance value. Cumulative measurement system 202 may further, at operation 712, update the cumulative measurement value to include the additional distance value. Once the cumulative measurement value has been updated and/or a position of an additional point has not been determined (e.g., no at operation 708), cumulative measurement system 202 may continue to determine whether positions of additional points have been determined on the anatomy (e.g., at operation 708). To illustrate, the cumulative measurement value may be dynamically updated as positions of additional points are determined to include additional distance values determined between sequential points of the additional points.

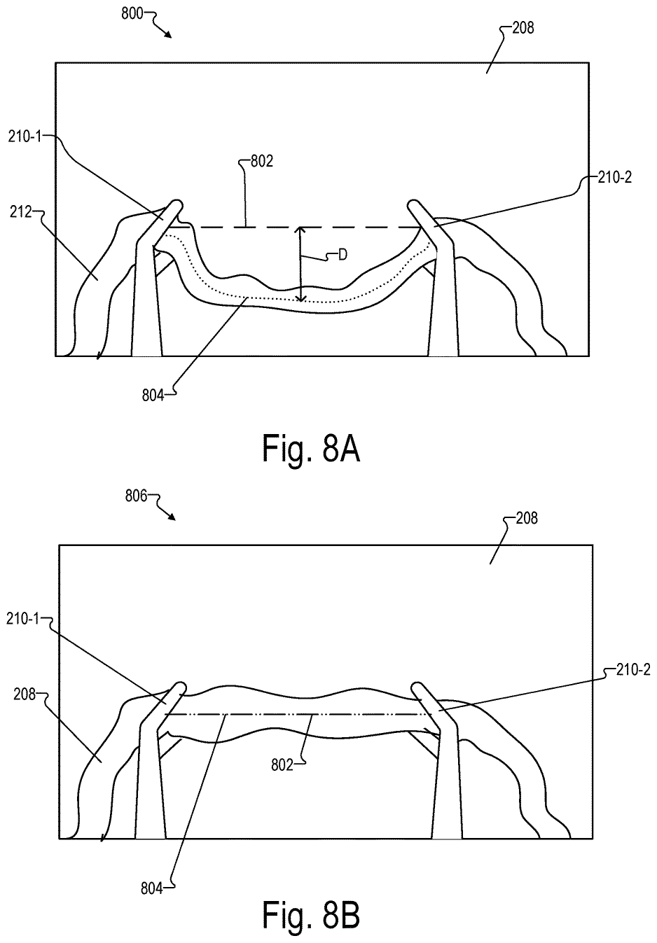

In some implementations, cumulative measurement system 202 may be configured to determine the positions of points and/or the distance values based on a straightness of the portion of the anatomy being held between the instruments. For example, FIG. 8A shows an illustrative implementation 800 of a display of scene 208 in which a portion of anatomy 212 is being held between instruments 210. As shown, the portion of anatomy 212 has slack between instruments 210 such that the portion of anatomy 212 is spaced a distance D away from an axis 802 formed between instruments 210. As the distance D is increased between anatomy 212 and axis 802 (e.g., a less amount of straightness), the determination of the distance value between points (e.g., points 404) on anatomy 212 between instruments 210 may become less accurate. Alternatively, as the distance D is decreased between anatomy 212 and axis 802 (e.g., a greater amount of straightness), the determination of the distance value between points (e.g., points 404) on anatomy 212 between instruments 210 may become more accurate.

In some implementations, the display of scene 208 may include a visual overlay of axis 802 formed between instruments 210 such that the visual overlay may indicate a level of straightness of the portion of anatomy 212 between instruments 210. To illustrate, the visual overlay may provide an indication of the distance D between anatomy 212 and axis 802 between instruments 210. In instances where the visual overlay of axis 802 is provided, cumulative measurement system 202 may dynamically update the visual overlay of axis 802 as one or both of instruments 210 move within scene 208.

Additionally or alternatively, the display of scene 208 may include a virtual overlay along a centerline 804 of the portion of anatomy 212 within scene 208 such that the virtual overlay of centerline 804 may indicate a level of straightness of the portion of anatomy 212. To illustrate, an amount of curve within the virtual overlay of centerline 804 may indicate an amount of straightness of the portion of anatomy 212. Additionally or alternatively, the virtual overlay of centerline 804 may be compared to the virtual overlay of axis 802 to indicate the level of straightness of anatomy 212. Moreover, the virtual overlay of centerline 804 may indicate an alignment of points positioned on the portion of anatomy 212. To illustrate, the positions of the points may be determined on centerline 804 of anatomy 212 such as to align the positions of the points on anatomy 212. Accordingly, the distance value between the positions of the points may be determined more accurately.

Additionally or alternatively, a straightness threshold may be used to indicate a minimum straightness sufficient for the portion of anatomy 212 between instruments 210 to determine a distance value associated with the portion of the anatomy. For example, cumulative measurement system 202 may determine the positions of points and/or the distance values based on detecting that the portion of anatomy 212 is straightened between instruments 210 within a threshold amount. To illustrate, cumulative measurement system 202 may determine when the distance D between anatomy 212 and axis 802 is within the threshold amount, which may indicate that anatomy 212 is sufficiently straight between instruments 210 such as to accurately determine the distance value. In some implementations, cumulative measurement system 202 may provide a straightness score based on the straightness of the portion of anatomy 212 relative to axis 802 that may be compared to the threshold amount. Additionally or alternatively, cumulative measurement system 202 may visually compare (e.g., using object recognition algorithms) the portion of anatomy 212 relative to axis 802 to determine whether the portion of anatomy 212 is straightened within the threshold amount.

As an illustrative example, FIG. 8B shows an implementation 806 of a display of scene 208 in which the portion of anatomy 212 between instruments 210 has been straightened. For example, instruments 210 may have been moved further apart to move the portion of anatomy 212 towards axis 802 between instruments 210 such as to align centerline 804 of anatomy 212 with axis 802. Accordingly, the distance D between anatomy 212 and axis 802 may be decreased to within the threshold amount, which may indicate that anatomy 212 may be sufficiently straight (e.g., satisfying the minimum straightness) between instruments 210 for determining positions of points and/or the distance value. While the illustrated implementation shows centerline 804 of anatomy 212 aligned with axis 802, centerline 804 may alternatively be offset from axis 802 within the threshold amount for anatomy 212 to be sufficiently straight between instruments 210.

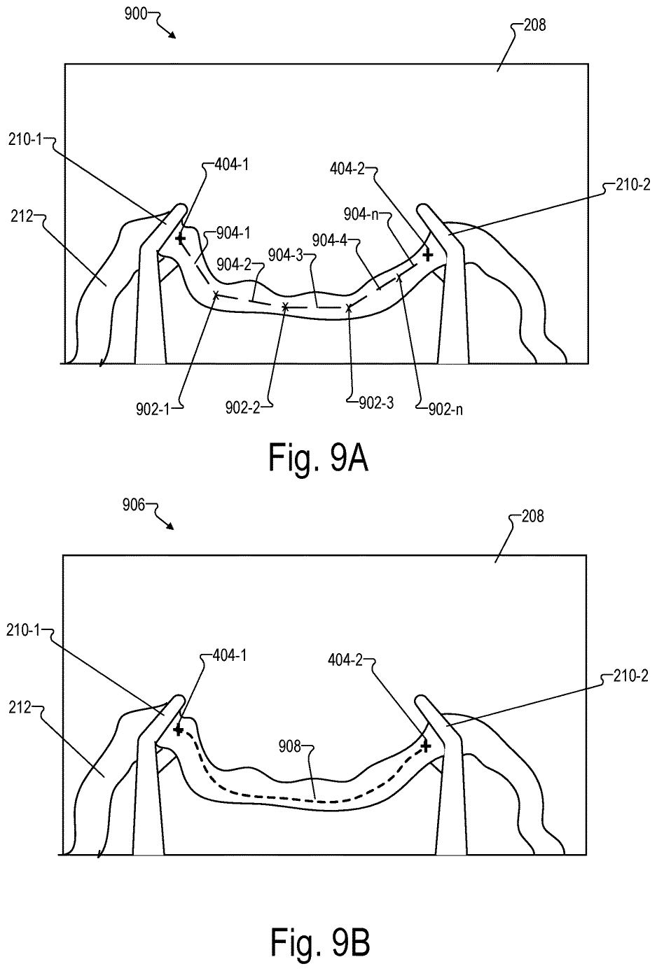

Alternatively, if the portion of anatomy 212 is not straightened between instruments 210 (e.g., anatomy 212 is outside the threshold amount), cumulative measurement system 202 may generate a plurality of segments to determine the distance value. As an illustrative example, FIG. 9A shows an implementation 900 of a display of scene 208 in which the portion of anatomy 212 is being held between instruments 210 such that the portion of anatomy 212 has not been straightened. As such, cumulative measurement system 202 may generate one or more intermediate points 902 (e.g., intermediate points 902-1 through 902-n) between points 404. Cumulative measurement system 202 may further generate one or more segments 904 (e.g., segments 904-1 through 904-n) between sequential intermediate points 902 and/or points 404 such that the distance value between points 404 may be calculated as sum of the distances of segments 904. In some implementations, the position and/or amount of intermediate points 902 may be adjusted based on a user input such that the distances of segments 904 may be adjusted.

Alternatively, the distance value between points 404 may be determined based on a contour (e.g., non-linear) distance between points 404. For example, the distance value may be determined when the curved portion of anatomy 212 being held between instruments 210 (e.g., between points 404) is visible from a viewpoint of the imaging device (e.g., imaging device 204). To illustrate, the curved portion of anatomy 212 may not be overlapped and/or coiled between points 404 such that the curved portion of anatomy 212 between points 404 is not obstructed within scene 208.

As an example, FIG. 9B shows an illustrative implementation 906 of a display of scene 208 depicting a contour 908 along the portion of anatomy 212 between points 404 such that the distance value may be determined based on a distance associated with contour 908. In some implementations, cumulative measurement system 202 may be configured to determine a distance associated with contour 908 by generating a deformable 3D model of anatomy 212 (e.g., based on images of scene 208). To illustrate, cumulative measurement system 202 may generate a point cloud having a plurality of nodes representative of surface points on at least the portion of anatomy 212 being held between instruments 210 within scene 208 and derive vertices associated with 3D locations that correspond to 3D locations of the plurality of nodes (e.g., using a SLAM heuristic). Cumulative measurement system 202 may associate vertices of the 3D model with the positions of points 404 and identify vertices along the 3D model between points 404 to determine contour 908. The distance associated with contour 908 may be determined based on distances between the 3D locations of the vertices along contour 908. Still other suitable methods may be used to determine the distance associated with contour 908. For example, a curve fitting algorithm (e.g., a polynomial function) may be used to fit contour 908 along anatomy 212.

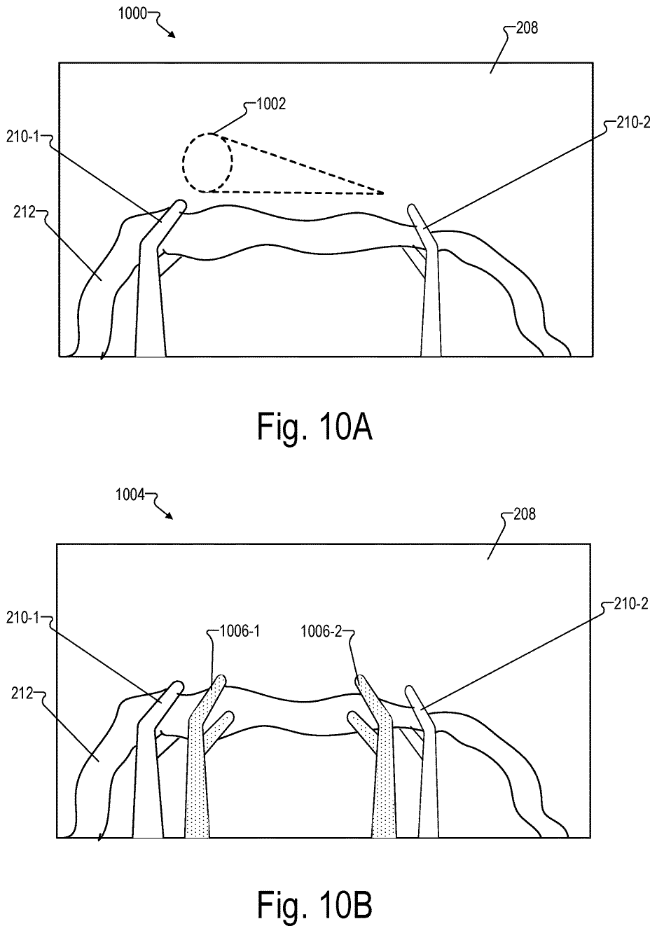

In some implementations, cumulative measurement system 202 may provide a visual depth indicator to indicate whether instruments 210 are aligned within scene 208 (e.g., for straightening anatomy 212 between instruments 210). For example, FIG. 10A shows an illustrative implementation 1000 of a display of scene 208 in which the portion of anatomy 212 is being held between instruments 210. The display further includes a visual depth indicator 1002 indicative of a depth between instruments 210 within a field of view of an imaging device (e.g., imaging device 204). In some implementations, visual depth indicator 1002 may include a 3D shape (e.g., a cone, a cylinder, a pyramid, a prism, etc.) extending between instruments 210 that may indicate a difference of the depth between instruments 210 within scene 208. To illustrate, visual depth indicator 1002 of the illustrated example is a cone having a larger volume near first instrument 210-1 and a smaller volume near second instrument 210-2, which may indicate that first instrument 210-1 is positioned closer to the imaging device within scene 208 than second instrument 210-2.

In some implementations, visual depth indicator 1002 may dynamically update as one or both of instruments 210 move within scene 208 relative to the imaging device. For example, as first instrument 210-1 and/or second instrument 210-2 move within scene 208 to align with each other at a depth relative to the imaging device, the shape of visual depth indicator 1002 may be dynamically updated with the movement of instruments 210. To illustrate, the volume of visual depth indicator 1002 may move toward second instrument 210-2 as the instruments 210 are aligned at the depth relative to the imaging device. When instruments 210 are aligned, visual depth indicator 1002 may have an equal volume near first instrument 210-1 and second instrument 210-2 such that visual depth indicator 1002 may form a cylinder. This may indicate that the depth of instruments 210 are aligned within scene 208. Still other suitable configurations for visual depth indicator 1002 may be used.

For example, FIG. 10B shows another illustrative implementation 1004 of a display of scene 208 that includes visual depth indicators 1006 (e.g., visual depth indicators 1006-1 through 1006-2). As shown, visual depth indicators 1006 include shadow images of each instrument 210 within scene 208. For example, a first visual depth indicator 1006-1 includes a shadow image of first instrument 210-1 and a second visual depth indicator 1006-2 includes a shadow image of second instrument 210-2 to indicate a target pose (e.g., corresponding to a target position and target orientation) for instruments 210 within scene 208 that may be used to align instruments 210 at a depth relative to the imaging device. To illustrate, first instrument 210-1 may be moved to a position within visual depth indicator 1006-1 and second instrument 210-2 may be moved to a position within visual depth indicator 1006-2 such as to align the depth of instruments 210.

In some instances, a target distance associated with anatomy may be desired (e.g., for a medical procedure) such that it may be desirable to measure a distance along anatomy 212 until the distance reaches the target distance. In such instances where a target distance is desired, cumulative measurement system 202 may be configured to compare the cumulative measurement value to a target measurement value representative of a target distance along anatomy 212. Based on the comparison, cumulative measurement system 202 may further be configured to determine when the cumulative measurement value corresponds to the target measurement value to indicate that the target distance has been measured along anatomy 212.

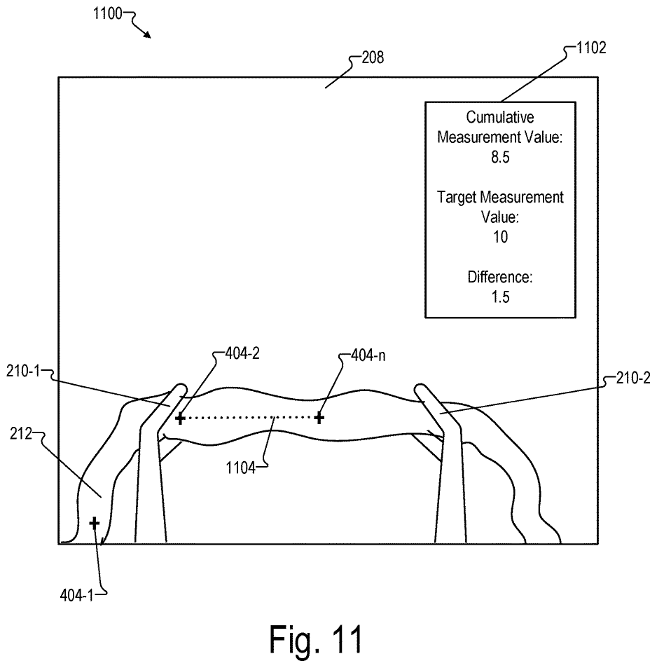

As an illustrative example, FIG. 11 shows an implementation 1100 of a display of scene 208 in which a portion of anatomy 212 is being held between instruments 210 for determining a target distance associated with anatomy 212. For example, it may be desirable to measure a distance associated with anatomy 212 to a target measurement value (e.g., “10”). Instruments 210 may be moved relative to anatomy 212 such as to designate positions of points 404 along the anatomy 212 for determining the cumulative measurement value (e.g., “8.5”). Cumulative measurement system 202 may update the cumulative measurement value as points 404 are added on anatomy 212 and compare the cumulative measurement value to the target measurement value to determine a difference (e.g., “1.5) between the cumulative measurement value and the target measurement value.

In some implementations, cumulative measurement system 202 may provide a legend 1102 in the display that may present the cumulative measurement value, the target measurement value, and/or the difference between the cumulative measurement value and the target measurement value. This may indicate when the cumulative measurement value corresponds to the target measurement value.

Additionally or alternatively, cumulative measurement system 202 may determine a position of an endpoint 404-n on anatomy 212 within scene 208 based on the difference between the cumulative measurement value and the target measurement value (e.g., to obtain the target measurement value). For example, cumulative measurement system 202 may determine the position of endpoint 404-n to be a distance on anatomy 212 from a previous point (e.g., second point 404-2) that is equal to the difference between the cumulative measurement value and the target measurement value. In some implementations, cumulative measurement system 202 may provide a marker on the display of scene 208 that may indicate the target position of endpoint 404-n to reach the target measurement value.

Still other suitable configurations may be used to indicate when the cumulative measurement value corresponds to the target measurement value. For example, the display of scene 208 may include a virtual overlay 1104 on anatomy 212 that may indicate the remaining distance to reach the target measurement value. Additionally or alternatively, cumulative measurement system 202 may provide a notification (e.g., visual, audio, etc.) to a user when the cumulative measurement value corresponds to the target measurement value.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

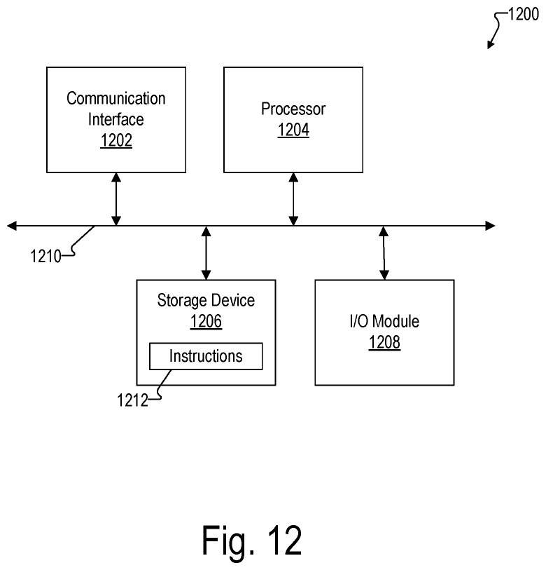

FIG. 12 shows an illustrative computing device 1200 that may be specifically configured to perform one or more of the processes described herein. Any of the systems, computing devices, and/or other components described herein may be implemented by computing device 1200.

As shown in FIG. 12, computing device 1200 may include a communication interface 1202, a processor 1204, a storage device 1206, and an input/output (“I/O”) module 1208 communicatively connected one to another via a communication infrastructure 1210. While an illustrative computing device 1200 is shown in FIG. 12, the components illustrated in FIG. 12 are not intended to be limiting. Additional or alternative components may be used in other embodiments. Components of computing device 1200 shown in FIG. 12 will now be described in additional detail.

Communication interface 1202 may be configured to communicate with one or more computing devices. Examples of communication interface 1202 include, without limitation, a wired network interface (such as a network interface card), a wireless network interface (such as a wireless network interface card), a modem, an audio/video connection, and any other suitable interface.

Processor 1204 generally represents any type or form of processing unit capable of processing data and/or interpreting, executing, and/or directing execution of one or more of the instructions, processes, and/or operations described herein. Processor 1204 may perform operations by executing computer-executable instructions 1212 (e.g., an application, software, code, and/or other executable data instance) stored in storage device 1206.

Storage device 1206 may include one or more data storage media, devices, or configurations and may employ any type, form, and combination of data storage media and/or device. For example, storage device 1206 may include, but is not limited to, any combination of the non-volatile media and/or volatile media described herein. Electronic data, including data described herein, may be temporarily and/or permanently stored in storage device 1206. For example, data representative of computer-executable instructions 1212 configured to direct processor 1204 to perform any of the operations described herein may be stored within storage device 1206. In some examples, data may be arranged in one or more databases residing within storage device 1206.

I/O module 1208 may include one or more I/O modules configured to receive user input and provide user output. I/O module 1208 may include any hardware, firmware, software, or combination thereof supportive of input and output capabilities. For example, I/O module 1208 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1208 is configured to provide graphical data to a display for presentation to a user. The graphical data may be representative of one or more graphical user interfaces and/or any other graphical content as may serve a particular implementation.

In the preceding description, various embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A computer-assisted medical system comprising:
a first repositionable manipulator arm coupled to a first instrument and configured to move the first instrument relative to an anatomy within a scene being imaged by an imaging device;
a second repositionable manipulator arm coupled to a second instrument and configured to move the second instrument relative to the anatomy within the scene; and
a computing device configured to perform a set of processes comprising:
determining, based on a position of the first instrument within the scene, a position of a first point on the anatomy;
determining, based on a position of the second instrument within the scene, a position of a second point on the anatomy;
determining, based on the position of the first point and the position of the second point, a first distance value representative of a physical distance between the first point and the second point;
identifying, based on an updated position of the second instrument, a position of a third point on the anatomy;
determining, based on the position of the third point, a second distance value representative of a physical distance between the third point and the second point;
tracking one or more of the first, second, or third points relative to the anatomy using a simultaneous localization and mapping heuristic, wherein the determining of at least one of the first or second distance values is further based on the tracking; and
determining, based on the first and second distance values, a cumulative measurement value representative of a distance along the anatomy between the first, second, and third points.

2. The computer-assisted medical system of claim 1, further comprising determining the positions of the first and second instruments based on at least one of kinematics data associated with the first and second instruments, a simultaneous localization and mapping heuristic, a depth map of the scene, or a machine learning algorithm.

3. (canceled)

4. The computer-assisted medical system of claim 1, wherein the determining the position of a select one or both of the first point or the second point is further based on a user input.

5. The computer-assisted medical system of claim 1, wherein the determining the position of the second point is performed prior to, subsequent to, or concurrently with the determining the position of the first point.

6. The computer-assisted medical system of claim 1, wherein the computing device is further configured to instruct a display device to display a plurality of visual markers within a display of the scene, wherein each visual marker of the plurality of visual markers is representative of a location of a select one of the first point, the second point, or the third point on the anatomy.

7. The computer-assisted medical system of claim 6, wherein the computing device is further configured to instruct the display device to display a virtual overlay between sequential visual markers of the plurality of visual markers within the display of the scene, wherein the virtual overlay is representative of a distance between the sequential visual markers.

8. The computer-assisted medical system of claim 1, wherein the computing device is further configured to instruct a display device to display a virtual overlay along a centerline of the anatomy within a display of the scene.

9. The computer-assisted medical system of claim 1, wherein the computing device is further configured to instruct a display device to display the first and second distance values.

10. The computer-assisted medical system of claim 1, wherein the determining the first and second distance values include detecting when the first instrument and the second instrument are holding a portion of the anatomy between the first instrument and the second instrument.

11. The computer-assisted medical system of claim 10, wherein the determining the first and second distance values is based on detecting that the portion of the anatomy is straightened between the first instrument and the second instrument within a threshold amount.

12. The computer-assisted medical system of claim 11, wherein the computing device is further configured to instruct a display device to display a visual overlay between the first instrument and the second instrument within a display of the scene, the visual overlay indicative of a straightness of the portion of the anatomy between the first instrument and the second instrument.

13. The computer-assisted medical system of claim 12, wherein the visual overlay is dynamically updated as one or both of the first instrument or the second instrument move within the scene.

14. The computer-assisted medical system of claim 10, wherein the determining the first and second distance values include generating segments along the anatomy between sequential points and calculating a sum of distances of the segments.

15. The computer-assisted medical system of claim 14, wherein the distances of the segments are adjustable based on user input.

16. The computer-assisted medical system of claim 10, wherein the determining the first and second distance values include generating contours along the anatomy between sequential points and determining distances associated with the contours.

17. The computer-assisted medical system of claim 10, wherein the first instrument and the second instrument are positioned to hold the portion of the anatomy such that the portion of the anatomy between the first instrument and the second instrument is visible within the scene.

18. The computer-assisted medical system of claim 1, wherein the computing device is further configured to instruct a display device to display a visual depth indicator within a display of the scene, the visual depth indicator indicative of a depth of the first and second instruments within a field of view of the imaging device.

19. The computer-assisted medical system of claim 18, wherein the visual depth indicator is dynamically updated as one or both of the first instrument or the second instrument move within the scene.

20-32. (canceled)

33. A method comprising:
determining, by a cumulative measurement system and based on a position of a first instrument within a scene, a position of a first point on an anatomy;
determining, by the cumulative measurement system and based on a position of a second instrument within the scene, a position of a second point on the anatomy;
determining, by the cumulative measurement system and based on the position of the first point and the position of the second point, a first distance value representative of a physical distance between the first point and the second point;
identifying, by the cumulative measurement system and based on an updated position of the second instrument, a position of a third point on the anatomy;
determining, by the cumulative measurement system and based on the position of the third point, a second distance value representative of a physical distance between the third point and the second point;
tracking one or more of the first, second, or third points relative to the anatomy using a simultaneous localization and mapping heuristic, wherein the determining of at least one of the first or second distance values is further based on the tracking; and
determining, by the cumulative measurement system and based on the first and second distance values, a cumulative measurement value representative of a distance along the anatomy between the first, second, and third points.

34-42. (canceled)

43. A non-transitory computer-readable medium storing instructions that, when executed, direct a processor of a computing device to perform a process comprising:
determining, based on a position of a first instrument within a scene, a position of a first point on an anatomy;
determining, based on a position of a second instrument within the scene, a position of a second point on the anatomy;
determining, based on the position of the first point and the position of the second point, a first distance value representative of a physical distance between the first point and the second point;
identifying, based on an updated position of the second instrument, a position of a third point on the anatomy;
determining, based on the position of the third point, a second distance value representative of a physical distance between the third point and the second point;
tracking one or more of the first, second, or third points relative to the anatomy using a simultaneous localization and mapping heuristic, wherein the determining of at least one of the first or second distance values is further based on the tracking; and
determining, based on the first and second distance values, a cumulative measurement value representative of a distance along the anatomy between the first, second, and third points.
